(19) United States
(12) Patent Application Publication
(10) Pub. No.: US 2025/0354200 A1
(43) Pub. Date: Nov. 20, 2025

(54) MULTIPLEXED AND SENSITIVE POINT-OF-CARE DIAGNOSTICS VIA INTEGRATED LATERAL FLOW AND IN-VITRO TRANSCRIPTION AND TRANSLATION

(71) Applicant: Georgia Tech Research Corporation, Atlanta, GA (US)

(72) Inventors: Alexandra Patterson, Atlanta, GA (US); Mark Styczynski, Atlanta, GA (US); Mike Farrell, Atlanta, GA (US)

(21) Appl. No.: 19/210,164

(22) Filed: May 16, 2025

Related U.S. Application Data
(60) Provisional application No. 63/648,271, filed on May 16, 2024.

(51) Int. Cl. C12Q 1/682; B01L 3/00

(52) U.S. Cl. CPC C12Q 1/682; B01L 3/5023; B01L 2300/069; B01L 2300/0825

(57) ABSTRACT

Disclosed herein. is a method of detecting the presence of a target, the method comprising: providing an in vitro transcription and/or translation area comprising a plurality of regions, each region comprising at least one distinct immobilized capture strand, each of the distinct immobilized capture strands configured to bind to a predetermined tail strand; introducing one or more mobile tail strands to the in vitro transcription and/or translation area, the one or more tail strands generated due to the presence of a corresponding target; introducing an in vitro transcription and/or translation reagent to the in vitro transcription and/or translation area; and producing one or more reporters indicative of a presence of one or more targets.

### CROSS-REFERENCE TO RELATED APPLICATIONS

This application claims the benefit of U.S. Provisional Application Ser. No. 63/648,271 filed on 16 May 2024, which is incorporated herein by reference in its entirety as if fully set forth below.

### GOVERNMENT LICENSE RIGHTS

This invention was made with government support under N66001-22-C-4012 awarded by the Defense Advanced Research Projects Agency. The government has certain rights in the invention.

### FIELD OF THE DISCLOSURE

The various embodiments of the present disclosure relate generally to systems and methods of an at-home diagnostic testing device, and more particularly to multiplexed at-home diagnostic testing device with lateral flow assay.

### BACKGROUND

The global pursuit of universal access to healthcare, as emphasized by the United Nations, is critically hindered by the limited availability of effective diagnostic tools, particularly in resource-limited settings. Conventional point-of-care (POC) diagnostic platforms, while successful in detecting simple biomarkers such as those for pregnancy or COVID-19, often fail to meet the demands of more complex diagnostic requirements. This is at least partly due to their limited multiplexing capabilities, which typically allow for the detection of only a few targets simultaneously, necessitating the use of expensive equipment and complex procedures that are not feasible for low-cost, accessible healthcare solutions.

Recent advancements in synthetic biology have introduced promising alternatives to traditional diagnostic systems, offering laboratory-grade capabilities and the potential for multiplexed detection. However, the transition of these technologies to POC applications has been stalled by the absence of low-resource, multiplexed reporters capable of indicating disease presence through simple, interpretable signals such as color changes. Accordingly, there is a need for an at-home detectors with a reporter configured to utilize multiplexing.

### BRIEF SUMMARY

A first aspect of the present disclosure provides a method of detecting the presence of a target, the method comprising: providing an in vitro transcription and/or translation area comprising a plurality of regions, each region comprising at least one distinct immobilized capture strand, each of the distinct immobilized capture strands configured to bind to a predetermined tail strand; introducing one or more mobile tail strands to the in vitro transcription and/or translation area, the one or more tail strands generated due to the presence of a corresponding target; introducing an in vitro transcription and/or translation reagent to the in vitro transcription and/or translation area; and producing one or more reporters indicative of a presence of one or more targets.

In any of the embodiments disclosed herein, the one or more reporters can produce a distinct color change, each color change indicative of the presence of a distinct target.

In any of the embodiments disclosed herein, the in vitro transcription and/or translation area can comprise a substrate, each capture strand can comprise a 3′ end and a 5′ end, and one of 3′ end and 5′ end can be bound to the substrate.

In any of the embodiments disclosed herein, the in vitro transcription and/or translation area can be a lateral flow assay.

In any of the embodiments disclosed herein, the distinct immobilized capture strands can be orthogonal to one another.

In any of the embodiments disclosed herein, the target can comprise one or more of a protein, a nucleic acid, and a small molecule.

In any of the embodiments disclosed herein, each capture strand can comprise a single stranded DNA sequence.

In any of the embodiments disclosed herein, each tail strand can comprise a single stranded DNA or RNA sequence.

In any of the embodiments disclosed herein, each tail strand can comprise a first region and a second region, the first region can comprise complementary nucleic acids to at least one capture strand, and the second region can comprise a functional region configured to generate an output.

In any of the embodiments disclosed herein, the output can comprise a material that is transcribable and/or translatable in an in vitro transcription and/or translation reaction.

In any of the embodiments disclosed herein, the second region can be configured to encode a material utilized to produce a reporter.

In any of the embodiments disclosed herein, the second region can be configured to capture a first material that encodes a second material utilized to produce a reporter.

A second aspect of the present disclosure provides a detection device, comprising an input and an in vitro transcription and/or translation area. The input can be configured to receive a plurality of mobile tail strands. Each of the plurality of tail strands can be generated due to the presence of a distinct target. The in vitro transcription and/or translation area can comprise a plurality of regions. Each region can comprise at least one distinct immobilized capture strand. Each of the distinct immobilized capture strands can be configured to bind to a predetermined tail strand to produce a reporter indicative of a presence of a distinct target.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides a schematic illustration of an example test sample in accordance with various embodiments of the present disclosure.

FIG. 2 provides a workflow of a multiplex detection method, in accordance with some embodiments of the present disclosure.

FIG. 3 provides a schematic illustration of multiplex detection method with an expanded detection step, in accordance with various embodiments of the present disclosure.

FIG. 4 provides a schematic illustration of multiplex detection method illustrating a capture/tail strand, in accordance with various embodiments of the present disclosure.

FIGS. 5A-B illustrate development of “barcodes” in which FIG. 5A illustrates orthogonality of 40 BP+Sapcer 5-plex designs (expression only on the diagonal represents minimal crosstalk) and FIG. 5B illustrates that the disclosed method induces expression only when target is present.

FIG. 6 illustrates the detection of multiplexed DNA targets using Rolling Circle Amplification (RCA) and an embodiment of the present disclosure.

FIGS. 7A-B illustrate embodiments of the present disclosure on LFA in which FIG. 7A illustrates capture and expression of DNA on LFA and FIG. 7B illustrates approximate color change expected from colorimetric enzyme.

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

As discussed above, there is a need for an at-home detection detectors with a reporter configured to utilize multiplexing. In response to this critical need, the present disclosure provides systems and methods allowing for POC multiplexed detection. Embodiments of this disclosure are generally referred to herein as the MEDIFLOW platform. MEDIFLOW stands for Multiplexed and Engineerable Diagnostics using Integrated FLOW, and it represents a significant advancement in POC diagnostics. In some embodiments, this innovative platform integrates expanded lateral flow assays (LFA) (though the disclosure is not limited to LFAs) with in vitro transcription and translation (IVTT) systems to achieve multiplexed, localized color-change reactions. This enables the sensitive and rapid detection of multiple targets (e.g., biomarkers) simultaneously, without the need for expensive equipment or complex sample preparation.

In some embodiments, MEDIFLOW leverages synthetic biology detection mechanisms, such as transcription factor-based sensors and rolling circle amplification, to produce unique single-stranded nucleic acids labeled with barcodes upon target detection. These reporter DNAs can then be added to an LFA, where they are captured on specific reporter zones through Watson-Crick base pairing. The captured sequences serve as templates for the transcription and translation of reporter enzymes, resulting in distinct color changes that correspond to the presence of different targets.

This approach not only facilitates multiplexing without the need for continual reengineering of the LFA but also eliminates the need for sample splitting, allowing all detection reactions to occur simultaneously in a single step. By addressing these challenges, MEDIFLOW offers a scalable, cost-effective solution for complex diagnostics at the point of care, significantly enhancing the accessibility and efficacy of healthcare diagnostics globally.

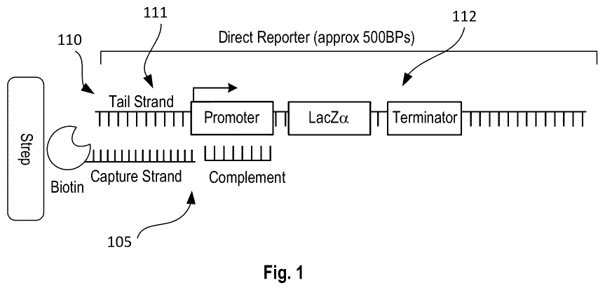

Widely used LFA diagnostics, such as the pregnancy test and Covid-19 test, use purified antibodies for protein detection via antigen-antibody binding on LFAs. Antigen-based LFAs are functional, but are time consuming to develop and can be challenging to multiplex. To overcome the need for antibodies, in some embodiments, MEDIFLOW captures single stranded nucleic acids generated by synthetic biology detection mechanisms (SBDM) via Watson-crick base pairing. Briefly, SBDM reactions occur in a multiplexed format where each target enables the synthesis of a unique single stranded “barcode.” The LFA can be separated into multiple reporter zones. Each reporter zone can contain tethered DNA complementary to one of the barcode sequences. The pooled SBDM reporters can flow up the LFA via capillary action and be captured on their cognate capture line via Watson-Crick base pairing, as shown in FIG. 1.

To sensitively visualize the single stranded captured barcodes, in vitro transcription and translation (IVTT) reactions can be used. IVTT is ideal for POC applications due to its low cost, shelf stability, and rapid response time. Furthermore, IVTT can be used to amplify signal, as one DNA can be transcribed repeatedly, one mRNA can be translated repeatedly, and one enzyme can cleave multiple substrates, resulting in exponential signal amplification. Specifically, IVTT reactions have been shown to detect as low as 1 aM of DNA. To enable integration of IVTT with SBDM, the SBDM product can be modified to encode for a reporter, such as a colorimetric enzyme, which when synthesized in an IVTT reaction, can produce a color change, e.g., from yellow to purple, as shown in (FIG. 1).

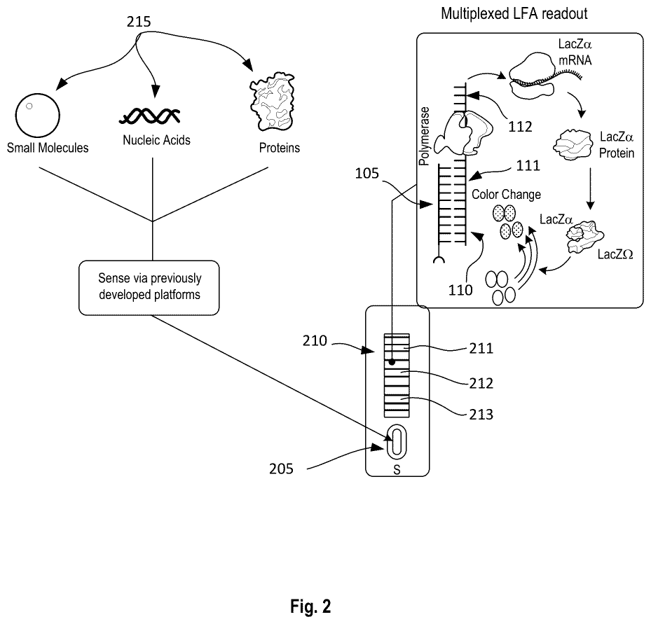

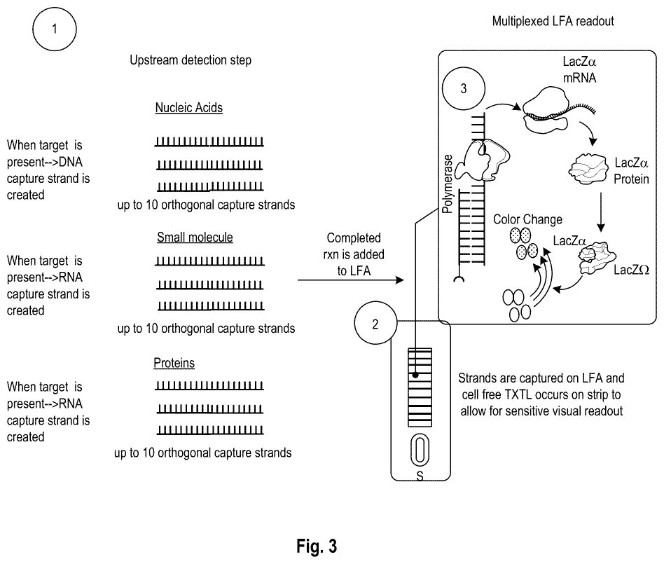

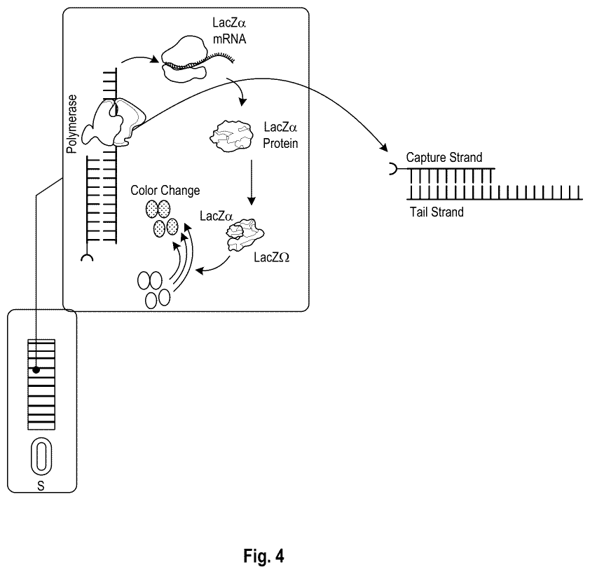

In summary, in some embodiments of the present disclosure, the diagnostic will be comprised of the following steps: 1) when target is present, single stranded nucleic acid barcodes will be synthesized; 2) the amplified barcodes will be added to an LFA and captured on their cognate line via DNA sequences complementary to the barcodes; 3) IVTT reagents will be added to the LFA; and 4) regions of the LFA with captured nucleic acids will change color, indicating target presence (FIGS. 2-4).

Various embodiments of the present disclosure and their features will now be discussed. In describing certain embodiments, the terms “capture strand” and “tail strand” are referenced below. FIG. 1 provides an exemplary embodiment of a tail 110 and capture 105 strand.

As used herein, the term “capture strand” (also referred to herein as “capture DNA”) generally refers to a single stranded DNA sequence that is immobilized through binding to a substrate of the IVTT area. Capture strands can have a 3′ end and a 5′ end, in which one of the ends is tethered to the substrate, thus immobilizing the capture strands. The capture strands can also be orthogonal to one another, i.e., the capture strands can exhibit low cross-binding and cross-reactivity and have minimal secondary (linear) structure.

As used herein, the term “tail strand” (also referred to herein as “tail DNA”) generally refers to a single stranded DNA or RNA sequence. As shown in FIG. 1, each tail strand 110 can have two or more regions. The first region 111 can have complementary nucleic acids to a corresponding capture strand 105. The second region 112 can be a functional region for generation of an output, including, but not limited to Green Flourescent Protein (GFP), LacZ color change enzyme, or any other output that can be transcribed and/or translated in an IVTT reaction. In some embodiments, the second region 112 can be configured to encode a material utilized to produce a reporter. In some embodiments, the second region 112 can be configured to capture one material that encodes another material utilized to produce a reporter. The captures strands 105 can be designed to specifically bind to the tail strand 110 via Watson-Crick base pairing, allowing for the capture and subsequent initiation of IVTT reactions, which result in a detectable color change on the IVTT area.

As used herein, the term “target” is used broadly to refer to any biomarker, biological molecule, or substance that can be detected and measured to indicate the presence or state of a disease or of a biological or non-biological process, condition, or disease, including pathogens or contaminants. Targets can be proteins, nucleic acids, metabolites, or other molecules that are indicative of normal or abnormal biological activity. In embodiments of the present disclosure, targets can be detected through synthetic biology mechanisms that synthesize or release unique single-stranded nucleic acids labeled with barcodes when the target, such as a pathogen, is present. These nucleic acids serve as reporters that can be captured and visualized through color-change reactions on a lateral flow assay, enabling rapid and sensitive diagnostics at the point of care.

FIG. 2 illustrates an exemplary multiplexed detection device in accordance with some embodiments of the present disclosure. The device can generally comprise an input 205 and an IVTT area 210. The input 205 can be configured to receive a one or more tail strands 110 generated due to the presence of a distinct target 215 in a sample. For example, a sample (e.g., blood, saliva, tissue, etc.) can be collected from a subject. Due to the presence of one or more targets 215 in the sample, certain tail strands 110 are produced. The substance with the tail strands 110 can then be placed in a sample region of the detection device, which can serve as an input 205. The input 205 can allow the mobilized tail strands 110 to enter the IVTT area 210.

The IVTT area 210 can comprise a plurality of regions 211 212 213. For example, in the embodiments shown in FIGS. 2-4, the IVTT area 210 comprises 10 regions, though any number of regions are contemplated within the scope of the present disclosure. Each region can comprise at least one distinct capture strand 105. As discussed above, each capture strand 105 can be immobilized on a substrate of the IVTT area 210 in the corresponding region. For example, a first region 211 can comprise a first type of capture strand 105, a second region 212 can comprise a second type of capture strand 105, and so on. Each type of capture strand 105 can be configured to bind (via Watson-Crick base pairing) to a predetermined tail strand 110 produced due to a specific target 215 in the sample. Once the corresponding capture 105 and tail 110 strands are bound, one or more IVTT reagents added to the IVTT area 210 can lead to IVTT reactions that produce a reporter indicative of the presence of the target 215 in the sample that led to the generation of that predetermined tail strand 110. In some embodiments, each reporter can produce a distinct color change, in which each color change can be indicative of the presence of the distinct target 215 in the sample.

Though the IVTT area shown in FIGS. 2-4 includes a lateral flow assay, the disclosure is not so limited, and other IVTT areas are contemplated. In particular, the IVTT area can be any area, such as a portion of a substrate, that allows for IVTT reactions to take place.

The example section below is provided to illustrate certain embodiments of the present disclosure but should not be construed a limited the scope of the claims appended hereto.

### EXAMPLES

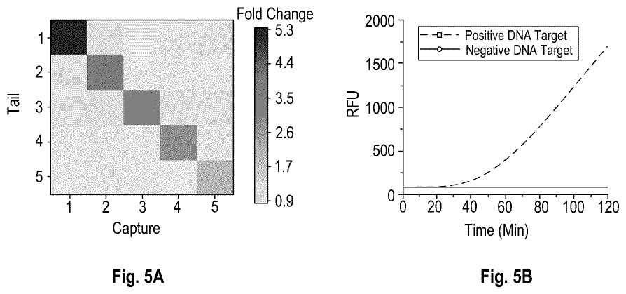

Preliminary Data: Short DNA sequences can be used to capture and initiate IVTT: DNA barcodes were designed in silico using NUPACK. Design constraints were enforced to ensure 1) no cross-reactivity between strands and 2) linearity of the single stranded barcodes (to increase speed of barcode-barcode binding). For ease of discussion, the barcode tethered to the LFA is referred to as the “capture strand” and the cognate barcode on the RCA product is referred to as the “tail DNA.” Initial testing of designed barcodes was completed in a plate-based assay to allow for high-throughput testing. Briefly, capture DNA was bound to a streptavidin plate. Tail DNA triggering expression of green fluorescent protein (GFP) in IVTT was added to capture DNA for 30 seconds (to mimic the short binding time of capture in the LFA). Any unbound tail was washed and removed. IVTT reagents were added to the captured DNA and GFP expression was measured. It was determined that the designed barcodes could specifically capture cognate tail DNA, and IVTT signal amplification could decrease the LOD by three orders of magnitude, enabling detection at only 0.005 pmols of tail (data not shown). Barcode design was optimized and a length of 40 plus a spacer (a short sequence on the capture strand that pushes the tail binding region away from the plate surface) was determined to be optimal (data not shown). The optimized design was scaled to 5-plex and verified to be orthogonal (see FIG. 5A).

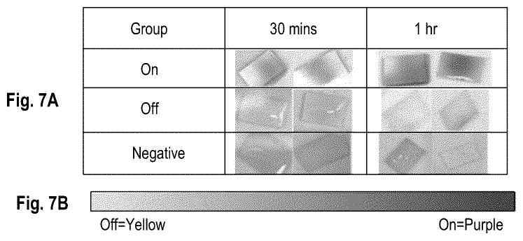

Designed barcodes can be captured and expressed on LFA: Two optimized capture sequences were conjugated to an LFA creating two test lines. Tail sequences were added to the bottom of the LFA and allowed to flow through both tethered capture sequences. After 10 minutes, LFAs were cut to separate the two test zones and IVTT reagents were added to initiate color change reactions. Optimized barcodes were shown to capture DNA on an LFA and initiate isolated IVTT reactions (see FIGS. 7A-B).

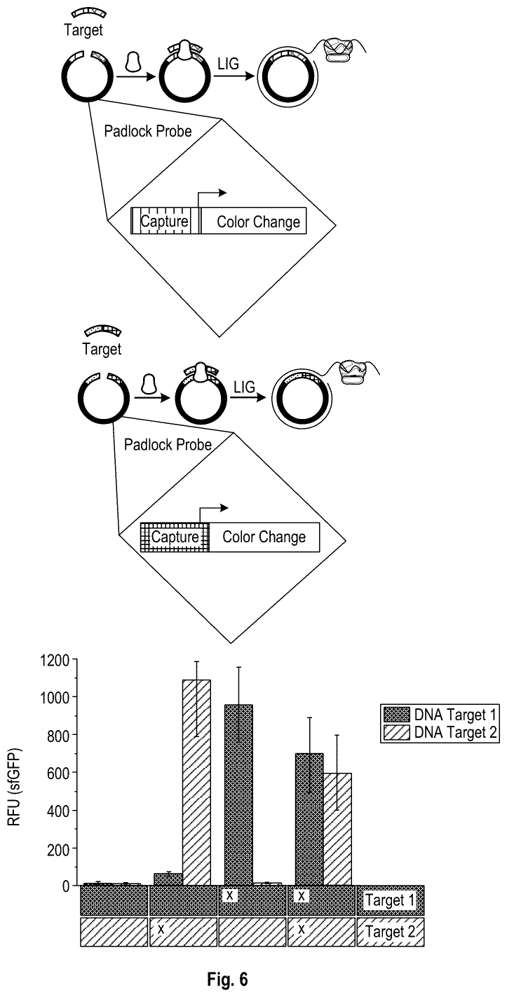

Integration of MEDIFLOW with SBDMs: MEDFLOW was integrated with rolling circle amplification to show detection of DNA. Here, 5′ and 3′ ends of a linear DNA probe are circularized via T4 DNA ligase when bound to a DNA target. Once circularized, rolling circle amplification using Phi29 DNA polymerase can generate long concatemers of the circularized probe. To modify RCA for MEDIFLOW, the IVTT barcodes developed above were integrated with RCA and padlock probe (RaPP). Briefly, a single stranded model padlock was modified to include a barcode (to capture) and an IVTT coding sequence (to initiate expression of GFP) (see FIG. 6). Probe and DNA target were chemically synthesized, and RaPP reactions were run according to previously developed protocols. The completed RaPP reactions were added to the plate-based assay described above (modified to have a 3 hr capture time). Excitingly, only when the DNA target was present was IVTT expression initiated (see FIG. 5B). Furthermore, this process was expanded to test for two DNA targets in a multiplexed format (see FIG. 6). When coupled with MEDIFLOW, RCA is able to be easily utilized for POC friendly multiplexing.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A method of detecting the presence of a target, the method comprising:
providing an in vitro transcription and/or translation area comprising a plurality of regions, each region comprising at least one distinct immobilized capture strand, each of the distinct immobilized capture strands configured to bind to a predetermined tail strand;
introducing one or more mobile tail strands to the in vitro transcription and/or translation area, the one or more tail strands generated due to the presence of a corresponding target;
introducing an in vitro transcription and/or translation reagent to the in vitro transcription and/or translation area; and
producing one or more reporters indicative of a presence of one or more targets.

2. The method of claim 1, wherein the one or more reporters produce a distinct color change, each color change indicative of the presence of a distinct target.

3. The method of claim 1, wherein the in vitro transcription and/or translation area comprises a substrate, and wherein each capture strand comprises a 3′ end and a 5′ end, wherein one of 3′ end and 5′ end is bound to the substrate.

4. The method of claim 1, wherein the in vitro transcription and/or translation area is a lateral flow assay.

5. The method of claim 1, wherein the distinct immobilized capture strands are orthogonal to one another.

6. The method of claim 1, wherein the target comprises one or more of a protein, a nucleic acid, and a small molecule.

7. The method of claim 1, wherein each capture strand comprises a single stranded DNA sequence.

8. The method of claim 1, wherein each tail strand comprises a single stranded DNA or RNA sequence.

9. The method of claim 8, wherein each tail strand comprises a first region and a second region, the first region comprising complementary nucleic acids to at least one capture strand, the second region comprising a functional region configured to generate an output.

10. The method of claim 9, wherein the output comprises a material that is transcribable and/or translatable in an in vitro transcription and/or translation reaction.

11. The method of claim 10, wherein the second region is configured to encode a material utilized to produce a reporter.

12. The method of claim 10, wherein the second region is configured to capture a first material that encodes a second material utilized to produce a reporter.

13. A detection device, comprising:
an input configured to receive a plurality of mobile tail strands, each of the plurality of tail strands generated due to the presence of a distinct target;
an in vitro transcription and/or translation area comprising a plurality of regions, each region comprising at least one distinct immobilized capture strand, each of the distinct immobilized capture strands configured to bind to a predetermined tail strand to produce a reporter indicative of a presence of a distinct target.

14. The device of claim 13, wherein each reporter produces a distinct color change, each color change indicative of the presence of a distinct target.

15. The device of claim 13, wherein the in vitro transcription and/or translation area comprises a substrate, each of the plurality of capture strands comprising a 3′ end and a 5′ end, wherein one of 3′ end and 5′ end is bound to the substrate.

16. The device of claim 13, wherein the in vitro transcription and/or translation area is a lateral flow assay.

17. The device of claim 13, wherein the distinct immobilized capture strands are orthogonal to one another.

18. The device of claim 13, wherein each capture strand comprises a single stranded DNA sequence.

19. The device of claim 13, wherein each tail strand comprises a single stranded DNA or RNA sequence.

20. The device of claim 13, wherein each tail strand comprises a first region and a second region, the first region comprising complementary nucleic acids to at least one capture strand, the second region comprising a functional region configured to generate an output.
